(19) United States
(12) Patent Application Publication
(10) Pub. No.: US 2024/0042072 A1
(43) Pub. Date: Feb. 8, 2024

(54) MODULAR IRRADIATION DEVICE AND IRRADIATION METHOD

(71) Applicant: FRAUNHOFER-GESELLSCHAFT ZUR FÖRDERUNG DER ANGEWANDTEN FORSCHUNG E.V., München (DE)

(72) Inventors: Bastian STANDFEST, Stuttgart (DE); Martin THOMA, Stuttgart (DE)

(73) Assignee: FRAUNHOFER-GESELLSCHAFT ZUR FÖRDERUNG DER ANGEWANDTEN FORSCHUNG E.V., München (DE)

(21) Appl. No.: 18/257,015

(22) PCT Filed: Dec. 15, 2021
(86) PCT No.: PCT/EP2021/085979

§ 371 Date: Jun. 12, 2023

(30) Foreign Application Priority Data
Dec. 16, 2020 (DE) 10 2020 216 088.0

(51) Int. Cl. A61L 2/00; A61M 1/36; A61M 99/00

(52) U.S. Cl. CPC A61L 2/0047; A61L 2/007; A61L 2/0088; A61M 1/36; A61M 99/00; A61L 2202/14; A61L 2202/22; A61M 2202/0423; A61M 2202/206; A61M 2202/097

(57) ABSTRACT

The invention relates to a modular irradiation device having a main module and at least one support cassette, the support cassette being insertable into a receptacle of the main module. The support cassette has at least one pump, and the main module has at least one pump actuator, these being arranged such that the pump is actuatable with the pump actuator when the support cassette is inserted into the receptacle of the main module.

The invention relates to a modular irradiation device comprising a main module and at least one carrier cassette, wherein the carrier cassette can be inserted into a receptacle of the main module. The carrier cassette comprises at least one pump, and the main module comprises at least one pump actuator, which are arranged in such a way that the pump can be actuated by way of the pump actuator when the carrier cassette is inserted into the receptacle of the main module.

Ionizing radiation is being increasingly used not only during the production of pharmaceuticals for novel therapies (advanced therapy medicinal products, ATMPs) and personalized medicine, but also during the inactivation and sterilization of, for example, pathogenic liquids. To ensure homogeneous irradiation, a constant dose must be applied to the liquid. During the irradiation with low-energy electron radiation, for example, the accelerated electrons lose energy with increasing penetration depth, so that the depth dose decreases. As a result, the liquid film to be treated should have a layer thickness of <200 μm. In addition to constant radiation parameters, constant flow properties of the liquid film are of importance, which encompass, amongst others, the layer thickness and the flow rate.

Another important aspect when processing ATMPs is the avoidance of cross-contamination. Especially with respect to the production of personalized medicine and/or the handling of low-volume patient specimens, the use of standardized disposables such as syringes, infusion bags and the like is advantageous.

DE102016216573A1 describes the irradiation of liquid thin films using a rotating stainless steel roller. Since this involves a solution that comes in contact with the product, a rapid change between different patient specimens that is free of cross-contamination cannot be ensured.

It is the object of the present invention to provide an irradiation device and an irradiation method that enable efficient irradiation of fluid samples with the lowest possible risk of cross-contamination.

The object is achieved by the modular irradiation device according to claim 1 and by the irradiation method according to claim 19. The respective dependent claims describe advantageous refinements of the modular irradiation device according to the invention and of the irradiation method according to the invention.

The invention relates to an irradiation device that has a modular design. The irradiation device comprises, as modules, a main module on the one hand and at least one carrier cassette on the other hand. Modules shall preferably be structural units, the components of which are in each case structurally connected to one another and can be handled together, without having to detach components of the modules.

According to the invention, the main module comprises at least one receptacle in which the at least one carrier cassette can be inserted so as to be removable without destruction. The carrier cassette can preferably be inserted into the receptacle without having to separate components of the carrier cassette from one another and without having to separate components of the main module from one another. The main module and at least one carrier cassette are thus preferably configured in such a way that the carrier cassette can be inserted into the receptacle of the main module in a state in which all components of the carrier cassette are connected to one another and in which all components of the main module are connected to one another, and without one or more elements of the carrier cassette having to be separated and without one or more elements of the main module having to be separated. The fact that the different modules, that is, in particular the main module and the carrier cassette, can be handled as a whole may be considered an expression of the modular concept of the invention.

According to the invention, the carrier cassette has at least one exposure surface on which at least one irradiation line runs. The term ‘exposure surface’ shall initially simply be interpreted as a surface area that can be irradiated by an irradiation source. In the simplest case, this could also only be the surface of the irradiation line itself. If the irradiation line, for example, is thus a hose, the hose surface facing the irradiation source could be considered to be the exposure surface. However, it is preferred when the exposure surface is a surface area into which the irradiation line is introduced.

According to the invention, a fluid to be irradiated can be conducted in the at least one irradiation line. The fluid can, for example, be a gas or, preferably, a liquid, wherein suspensions, for example of cells, can likewise be considered to be a liquid within the present meaning. The fluid line can be a hose or a channel, for example, wherein a channel is introduced into the exposure surface and can be covered by a film that at least partially covers the exposure surface.

According to the invention, the carrier cassette comprises at least one pump, which is connected via a first fluid line to the at least one irradiation line. A pump here may be understood to mean a device by way of which the fluid can be delivered. Preferably, those elements that act on the fluid are referred to as a pump, that is, in particular a plunger and a pump chamber, for example, however an actuator for actuating the pump shall not be considered to be part of the pump itself.

The at least one pump being connected via the first fluid line to the at least one irradiation line shall be understood to mean here that, as a result of the actuation of the pump, fluid can be moved through the first fluid line into the irradiation line or can be moved out of the irradiation line into the first fluid line. The pump is thus connected via the first fluid line to the at least one irradiation line in a fluid-conducting manner.

According to the invention, the main module comprises at least one receptacle in which the at least one carrier cassette can be inserted so as to be removable without destruction. Particularly preferably, the at least one carrier cassette can be inserted into the at least one receptacle without separating components of the main module and without separating components of the carrier cassette. The carrier cassette can thus preferably be inserted as a whole into the receptacle.

According to the invention, the main module additionally comprises at least one pump actuator, which is arranged so as to be able to actuate the at least one pump of the carrier cassette thereby when the corresponding carrier cassette is inserted into the corresponding receptacle. The process of inserting the carrier cassette into the receptacle can encompass multiple steps, such as, for example, the placing of the carrier cassette into the receptacle and the closing, for example the locking, or similar further steps. In particular, the process of inserting can encompass steps in which, for example, a pump coupling element of the pump actuator is made to engage with the pump or an actuatable element of the pump. For example, inserting connecting elements between the actuator and the corresponding pump can also be understood to be part of the insertion process. If these connecting elements are not connected to the main module or the carrier cassette, they would not be considered to be part of either of the two modules. The actuatability of the pump by the pump actuator should exist at the latest when all steps of inserting the carrier cassette into the receptacle have been completed.

A large number of different options exist for arranging the at least one pump in the carrier cassette and the at least one pump actuator at the main module so that the pump can be actuated by way of the pump actuator when the carrier cassette is inserted into the receptacle of the main module. The exact embodiment depends on the embodiment of the pump as well as on the embodiment of the pump actuator. If, for example, the pump is configured as a syringe, the pump actuator can have a pressure surface, for example, which becomes seated against an end surface of a syringe plunger of the syringe when the carrier cassette is being inserted into the receptacle, so that the pump actuator can push with the pressure surface onto the end face of the syringe plunger and thereby push the syringe plunger into the syringe cylinder. As an alternative or in addition, the pump actuator can, for example, also comprise a gripper element, which can rest behind the end surface of the pump plunger against the rear side thereof when the cassette is inserted into the receptacle, so that the pump actuator can pull the plunger out of the syringe cylinder. These embodiments, however, shall only be understood to serve as examples. For a given form of pump, a person skilled in the art will always be able to select the actuator accordingly and to arrange the actuator so as to be able to actuate the pump.

In an advantageous embodiment of the invention, the carrier cassette can comprise at least one further of the pumps, which is connected via a second fluid line to the irradiation line. In particular, such a further pump can advantageously be arranged at an opposite end of the irradiation line, opposite the first pump. In this way, the fluid can be conducted from the one pump through the irradiation line to the other pump. It is sufficient when only one of the pumps is actuated or only the pumps on one side of the irradiation line are actuated. The pumps on the other side then only act as provision or receiving containers and are moved by the fluid itself. An embodiment in which those pumps in which the fluid is moved through the irradiation line are actuated is advantageous. In this case, the fluid is thus suctioned through the irradiation line. It is advantageous in the process that the fluid cannot exit the system through leaky spots. It is also possible to transport the fluids using positive pressure.

In an advantageous embodiment of the invention, the pump actuator can comprise a pump coupling element, which is arranged so as to become engaged with a movable element of the corresponding pump in a form-locked manner when the cassette is being inserted into the main module. Form fit is thus created between the pump coupling element and the movable element of the pump, by way of which the pump actuator can exert a force or torque on the movable element of the pump. In the simplest case, the pump coupling element can be a bearing surface or pressure surface, which can push on the movable element of the pump when the actuator is active. However, the movable element of the pump can also comprise a protrusion, for example, wherein the pump coupling element can then, for example, comprise a projection or a fork, which engages behind the protrusion in such a way that the movable element of the pump can be actuated in the direction of the protrusion. It is also possible, for example, for the pump coupling element and the movable element of the pump to form a joint, such as, for example, a dovetail joint, when the carrier cassette is being inserted into the receptacle of the main module.

In an advantageous embodiment of the invention, the at least one carrier cassette can comprise at least one valve, which is arranged in at least one of the fluid lines and by way of which a fluid flow between the corresponding pump, which is connected via this fluid line to the irradiation line, and the irradiation line can be controlled. The valve being arranged in the fluid line shall be understood to mean that the valve is arranged at one end of the fluid line or is connected between two parts of the fluid line, wherein a portion of the fluid line is then arranged at one port of the valve, and the other portion of the fluid line is arranged at another port of the valve. The flow of the fluid through the fluid line thus takes place through the valve.

In this embodiment, the main module preferably comprises a respective valve actuator for one, several or all of the valves, the valve actuators being in each case arranged so as to be able to adjust the corresponding valve when the carrier cassette is inserted into the corresponding receptacle of the main module. In this way, it is possible, by inserting the carrier cassette into the receptacle, to create a state in which the valves can be actuated by the valve actuators. The valve actuators can then advantageously be automatically controlled so that the fluid flow in the carrier cassette can be automatically controlled via the valves.

The at least one pump actuator and/or the at least one valve actuator can advantageously be electrical, pneumatic, hydraulic or magnetic actuators.

An embodiment in which the at least one valve comprises a stopcock is advantageous, by way of which the valve can be adjusted for controlling the flow of fluid. In this case, the corresponding valve actuator, which comes in contact with this valve when the carrier cassette is being inserted into the receptacle, can be coupled to the stopcock in such a way that a force adjusting the valve or a torque adjusting the valve can be exerted on the stopcock by way of the valve actuator. Advantageously, form fit can arise between the coupling element and the stopcock of the corresponding valve when the carrier cassette is being inserted into the receptacle. Advantageously, the coupling element can then become engaged with the stopcock of the corresponding valve when the carrier cassette is being inserted into the main module. Again, numerous options exist as to how the valves can be coupled with the stopcocks thereof to the coupling elements. This embodiment of the invention is not limited to a certain form of the coupling or a certain arrangement. For example, the valves can be arranged in the carrier cassette in such a way that the valves are arranged on the outside of the carrier cassette, and the valve stopcocks are directed away from the carrier cassette to the outside. Valve actuators can then be arranged at the main module so as to surround an area in which the carrier cassette is present when inserted into the receptacle. If, in the inserted state, the valve stopcock is in each case arranged at the same height and in the same direction as the corresponding actuator, the valve stopcock and the actuator can become engaged with one another. This embodiment, however, shall only be understood as an example, and other arrangements are readily possible.

In an advantageous embodiment of the invention, at least one of the at least one valves can be a three-way valve, which has three ports. One of the three ports can then advantageously be connected to one of the fluid lines, which open into the irradiation line, and the other two ports can each be connected to a pump. In this way, the three-way valve allows switching between a state in which one pump is connected to the irradiation line and a state in which the other pump is connected to the irradiation line. It is thus possible for multiple pumps to be provided in the carrier cassette, which include, for example, differing fluids which can be conducted through the irradiation line at different times and between which it is possible to switch by positioning the valve.

The invention can generally be implemented with any type of pump. However, an embodiment in which the at least one pump comprises a fluid chamber and a plunger is preferred, wherein the plunger seals the fluid chamber in a fluid-tight manner and can be displaced in the fluid chamber. In particular, the fluid chamber can advantageously be cylindrical, and the plunger can have an end face with which the plunger bounds the fluid chamber and the shape of which is essentially identical to the base surface of the fluid chamber. The respective pump actuator can engage on the plunger of the pump when the at least one carrier cassette is inserted into the corresponding receptacle. As a result, a force can be effectuated in a displacement direction of the plunger by way of the pump actuator.

In an advantageous embodiment, the pump can be a syringe, and particularly preferably an exchangeable plastic syringe. In this way, it becomes possible to accommodate the fluid or fluids in the corresponding syringe prior to or after irradiation, and to dispose of the syringes after use. As a result, the sterility of the system can be established since the syringes can be disposed of as contaminated parts.

In a particularly advantageous embodiment of the invention, the carrier cassette can comprise two pumps that are connected to the first fluid line, and three pumps that are connected to the second fluid line. In this embodiment, for example, fluids to be moved through the irradiation line can be provided in the three pumps connected to the second fluid line, and the two pumps connected to the first fluid line can serve as a receiving vessel for irradiated fluid on the one hand, and as a receiving vessel for waste products on the other hand.

In an advantageous embodiment, a disinfecting agent, for example, can be provided in the first of the pumps connected to the second fluid line, a cell medium can be provided in the second of the three pumps, and a cell suspension can be provided in the third of the three pumps. It is then possible, for example by way of a three-way valve, to connect the pump including the disinfecting agent to the irradiation line, and to pump the disinfecting agent through the irradiation line. On the side of the first fluid line, it is possible, for example likewise by way of a three-way valve, for the syringe for waste products to be connected to the irradiation line. The disinfecting agent is then delivered through the irradiation line into the pump for waste products. On the side of the second fluid line, it is then possible, for example by way of a three-way valve, for the syringe including cell medium to be connected to the irradiation line, and for the cell medium to be transported through the irradiation line. This cell medium can also be delivered, for example, into the pump for waste products on the side of the first fluid line. On the side of the second fluid line, it is then possible for the pump including cell suspension to be connected to the irradiation line, and for the cell suspension to be delivered through the irradiation line, which can then be exposed to irradiation, for example. On the side of the first fluid line, the pump for processed fluid can then be connected to the irradiation line so that the irradiated cell suspension is delivered into this pump.

In an advantageous embodiment of the invention, the main module can include a main module surface, which is arranged with respect to the receptacle in such a way that the main module surface and the exposure surface of the carrier cassette are coplanar when the carrier cassette is inserted into the corresponding receptacle of the main module. As a result of this main module surface, radiation from the irradiation source can be prevented from impinging on other elements of the main module and/or of the carrier cassette which are not to be exposed to the irradiation. The exposure surface and the main module surface are preferably configured in such a way that only the irradiation line is exposed to the irradiation. Advantageously, the exposure surface of the carrier cassette can, preferably completely, fill in an opening in the main module surface so that the inner edge of this opening rests against the outer edge of the exposure surface.

In a particularly advantageous embodiment, the main module comprises exactly one actuator for each of those pumps that are provided for receiving fluid that has passed through the irradiation line. In this embodiment, no actuators are provided for those pumps in which fluid is provided so as to be conducted through the irradiation line. In this embodiment, the fluid transport is effectuated in that the fluid is suctioned through the irradiation line. This is advantageous since the egress of fluid through potentially leaky spots is prevented.

In an advantageous embodiment of the invention, the main module can comprise two foldable side parts, by way of which the receptacle for the at least one carrier cassette can advantageously be closed. The corresponding carrier cassette can thus initially be inserted into the receptacle, and the process of inserting can be continued or completed by folding the foldable side parts into a closed position. Particularly advantageously, the foldable side parts can comprise several or all of the actuators and/or pump actuators. It is particularly preferred here when those actuators by way of which the pumps and/or valves that are connected via the first fluid line to the irradiation line can be actuated are arranged at one of the side parts, and those actuators by way of which the pumps and/or valves that are connected via the second fluid line to the irradiation line can be actuated are arranged at the other of the side parts. This arrangement is in particular useful when the pumps connected to the first and second fluid lines are arranged on opposite halves of the carrier cassette. In the closed state, the two side parts can then meet at a plane that intersects, and preferably cuts in half, the irradiation line, and that is situated between the pumps connected to the first fluid line and the pumps connected to the second fluid line.

In an advantageous embodiment of the invention, the carrier cassette can comprise a fluid chip. This fluid chip can comprise a base body, which has a base body exposure surface in which the at least one irradiation line can be embodied. The base body exposure surface can provide the exposure surface of the carrier cassette or be part of this exposure surface. The at least one irradiation line can then advantageously be a channel or a channel structure including at least one channel. The base body can comprise a first fluid connection to which the first fluid line is connected, and a second fluid connection to which the second fluid line is connected. The fluid connections can be configured in any possible way, for example as plug connections; however, this embodiment shall also encompass an embodiment in which the base body and the first and/or second fluid lines have a monolithic design. The fluid connection here would simply be the transition between the corresponding fluid line and the channel structure.

The base body can additionally comprise a film, which is arranged on the base body exposure surface and covers the channel structure. The film can cover the channel structure so as to seal the same against the egress of fluid onto the base body exposure surface. The film is preferably present on the exposure surface at least in those areas where the channel structure is formed.

For example, a polyethylene block can serve as the base body, which can be injection-molded, for example. The film can, for example, comprise or be a PET-PE film or the like.

In an advantageous embodiment, the channel structure can include a multitude of the channels, which converge at the respective ends thereof in the respective fluid connection. The channels can thus converge with one of the ends thereof at the one of the fluid connections, and can converge with the other ends thereof at the other of the fluid connections. An embodiment in which the channels converge in each case in pairs at the ends thereof in shared channels, and the shared channels, in turn, converge in each case in pairs in shared channels until exactly two shared channels converge in one of the fluid connections is particularly advantageous. Proceeding from the respective fluid connection, a tree structure of the channels may result in the process, in which a respective channel splits into two channels until the splitting channels open into long channels that extend over the exposure surface and, on the other side, in turn open into a tree structure, preferably having the same design, in which these are combined again to the other fluid connection. The long sections of the channels preferably extend in a straight manner and parallel to one another.

The base body can preferably be a monolithic block, for example an injection-molded part, into which the channel structure is embossed and/or cut. Cutting shall be understood to mean any machining operation, that is, for example, carving, engraving, milling, and the like.

The film can advantageously have a thickness of ≤80 μm, preferably ≤60 μm and/or of ≥1 μm, preferably ≥10 μm. The film can advantageously be a polyethylene film, for example.

The at least one channel can advantageously be embossed into the base body of the fluid chip to a depth of ≤300 μm, preferably ≤200 μm and/or preferably ≥10 μm, preferably ≥50 μm. These depths are in particular advantageous for irradiation with low-energy electron radiation since it is ensured at these depths that the fluid flowing in the channel is exposed to the irradiation over the entire depth.

It is advantageous to produce the fluid chip by means of channels that are embossed into a base body and sealed off by a film since this structure can be cost-effectively produced as a disposable element.

The invention furthermore relates to an irradiation method for irradiating a fluid. A modular irradiation device is used in the process, such as was described above. The irradiation method provides that at least one of the at least one carrier cassettes of the modular irradiation device is inserted into the main module and then, optionally thereafter, optionally immediately thereafter, the exposure surface of the at least one carrier cassette irradiated with ionizing radiation, while at the same time a fluid to be irradiated is moved through the at least one irradiation line and then, optionally thereafter, optionally immediately thereafter, the carrier cassette is removed from the main module.

This procedure is advantageous since it is very efficient. It is possible to prepare the carrier cassette by filling the corresponding pumps with fluid, then inserting it into the main module in a step of short duration, carrying out the irradiation and the fluid transport, and removing the carrier cassette again. Advantageously, a plurality of carrier cassettes can be used, which can then be individually prepared and consecutively inserted into the main module in rapid succession to carry out the irradiation. Particularly advantageously, the carrier cassettes can be completely designed as disposable elements since they do not contain the complex actuators. In this way, they can be disposed of without having to be sterilized after the processed fluids have been removed. Since the fluid never comes in contact with the main module, cross-contamination between fluids in different carrier cassettes can be precluded. A large number of separate fluid samples can be efficiently irradiated in this way.

If multiple carrier cassettes are provided, the method can thus be carried out in such a way that at least one further carrier cassette is inserted into the main module once or several times after the respective preceding carrier cassette has been removed from the main module, and then the exposure surface of the at least one further carrier cassette is irradiated with ionizing radiation, while a fluid to be irradiated is moved through the at least one irradiation line of this further carrier cassette.

A particularly advantageous procedure can provide that a disinfecting agent is moved through the irradiation line after the at least one carrier cassette was inserted into the main module, and before the fluid to be irradiated is moved through the irradiation line. In this way, it can be ensured that the fluid to be irradiated remains sterile during irradiation.

An advantageous procedure can additionally provide that a cell medium is moved in and/or through the irradiation line after the at least one carrier cassette was inserted into the main module, and before the fluid to be irradiated is moved through the irradiation line, and/or after the fluid to be irradiated was moved through the irradiation line. In particular, it is advantageous when the cell medium is moved through the irradiation line subsequent to a potential disinfecting agent since in this way the fluid to be irradiated can be prevented from becoming mixed with disinfecting agent.

It is advantageous when the fluid transport through the irradiation line is effectuated in that the corresponding of the pumps generate negative pressure. The fluid is thus suctioned in each case through the irradiation line. As a result, inadvertent egress of fluid can be prevented.

The method according to the invention is particularly advantageous for irradiating cell suspensions, virus suspension, medium, serum and/or blood samples. The fluid to be irradiated can thus advantageously contain or be a cell suspension.

Particularly advantageously, ionizing radiation, in particular electrons and/or UV radiation, may be used for irradiation. Accordingly, the irradiation source can be an electron source or a UV radiation source.

The invention will be described hereafter by way of example based on several figures. The features shown in the figures can also be implemented independently of the corresponding example and be combined with one another in the various examples.

In the drawings:

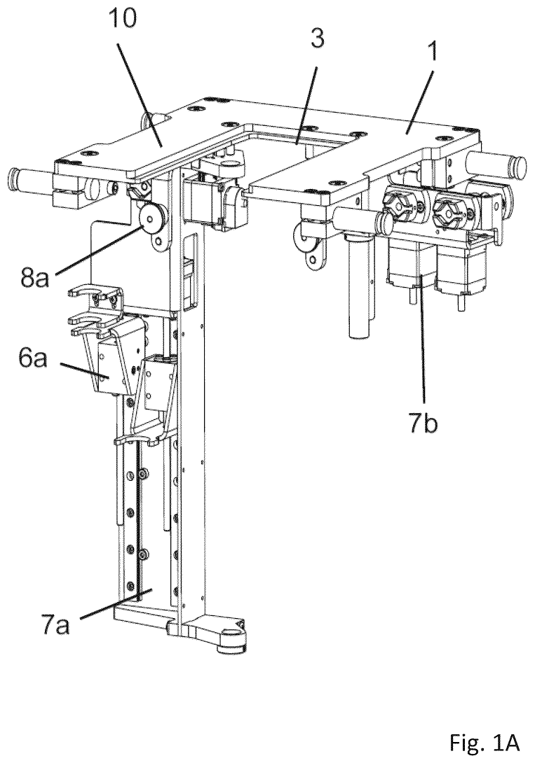

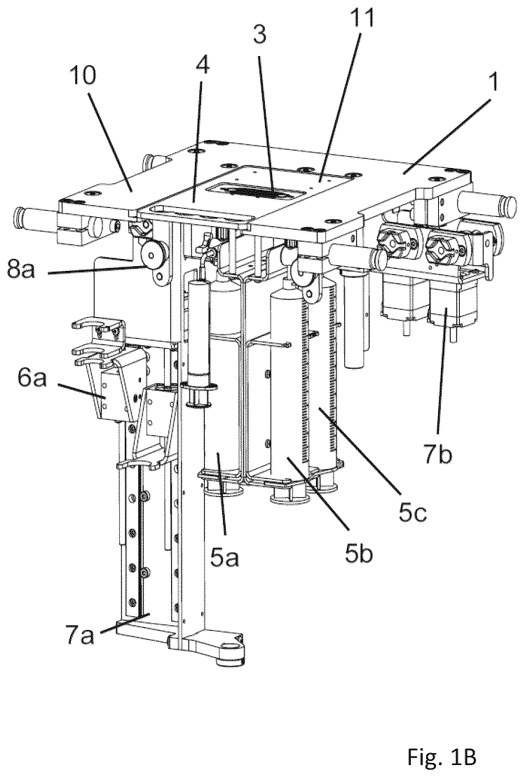

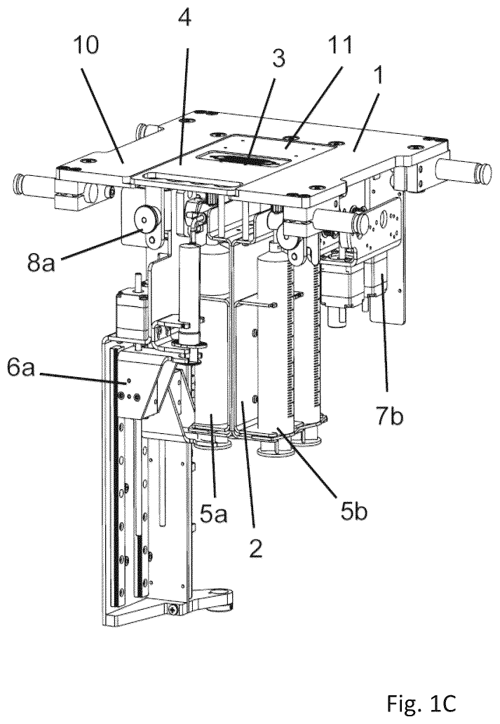

FIGS. 1A, B, C show a modular irradiation device;

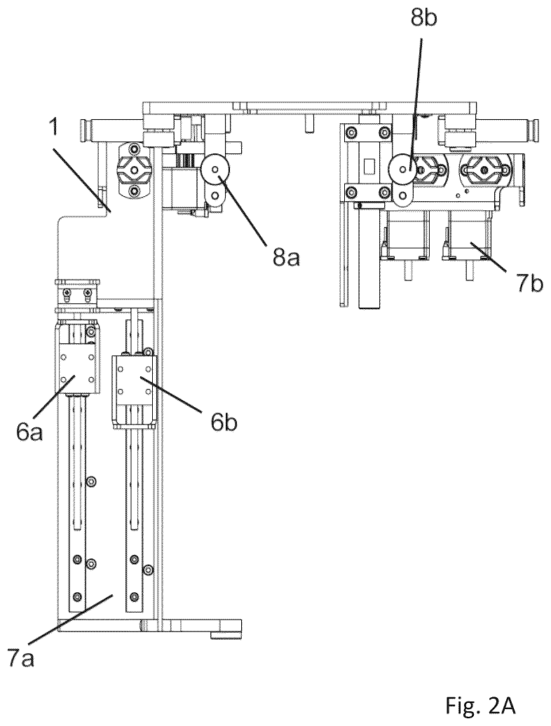

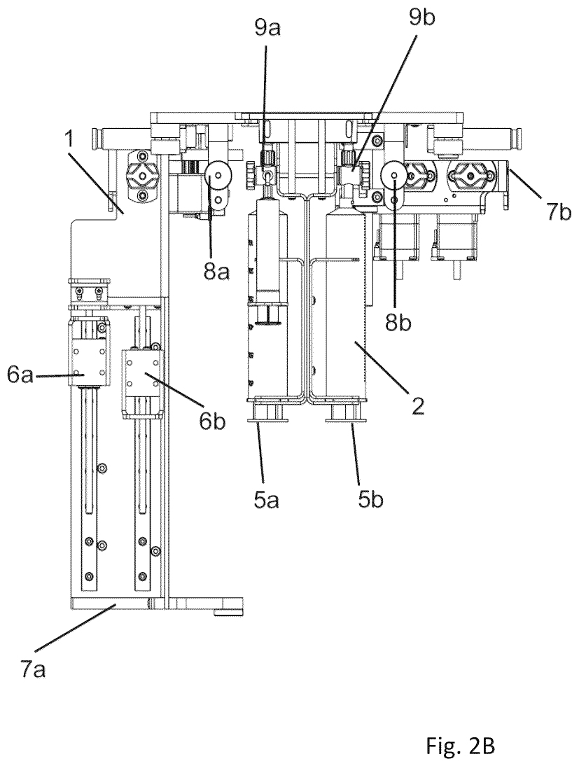

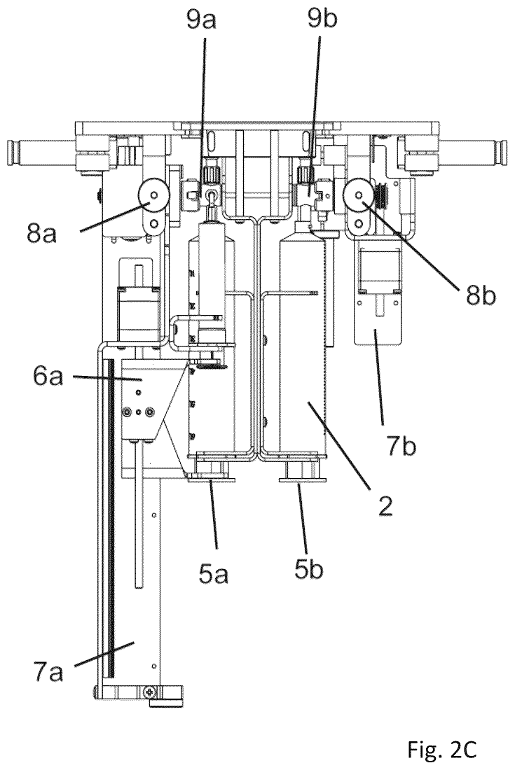

FIGS. 2A, B, C show a modular irradiation device;

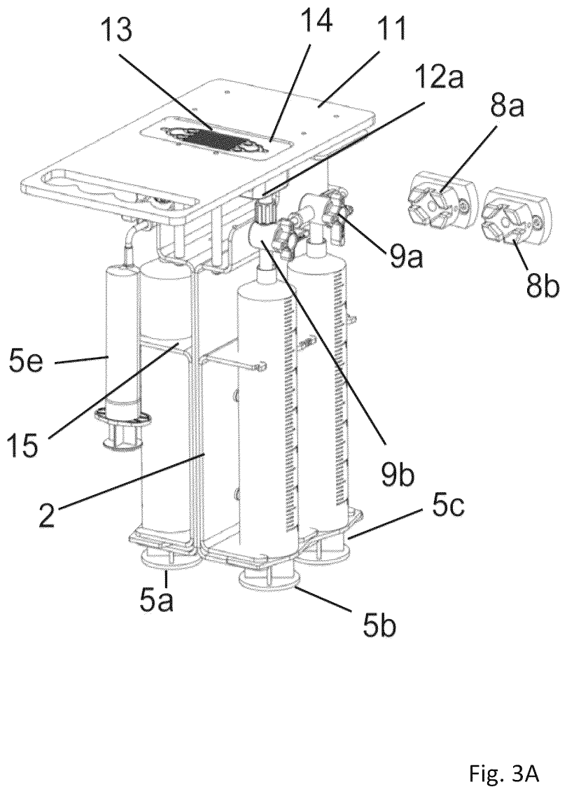

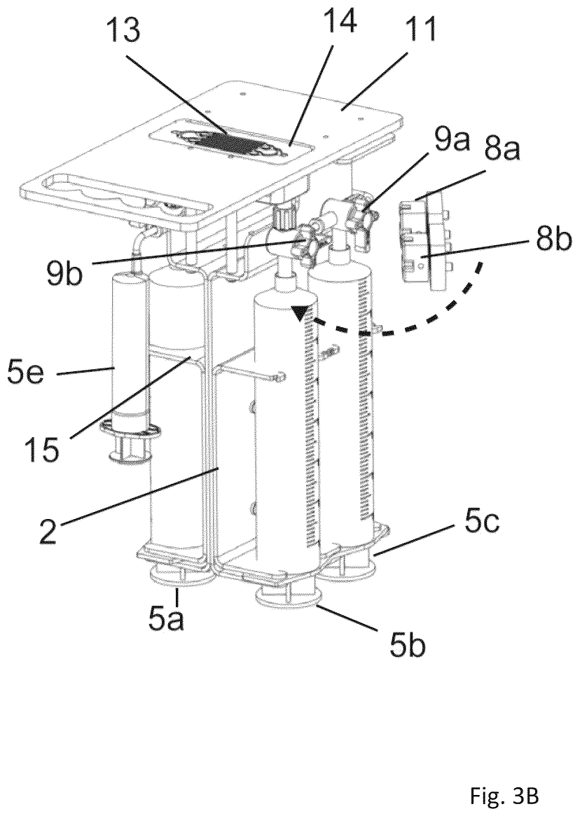

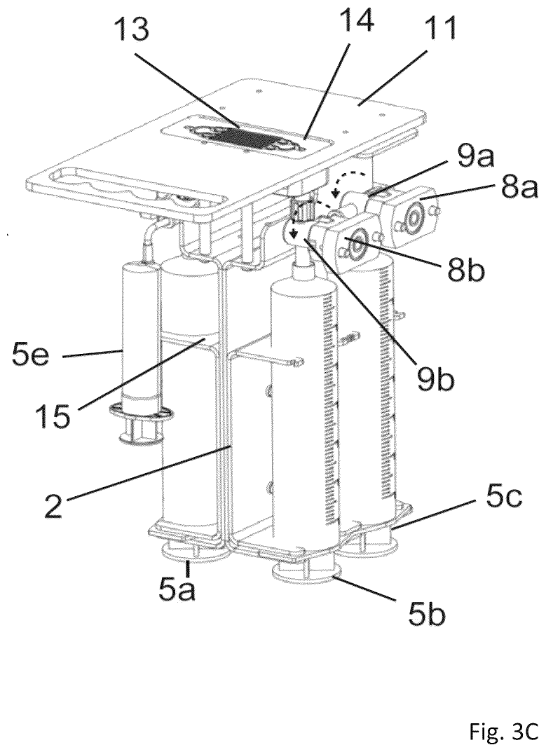

FIGS. 3A, B, C show a carrier cassette including some elements of a main module;

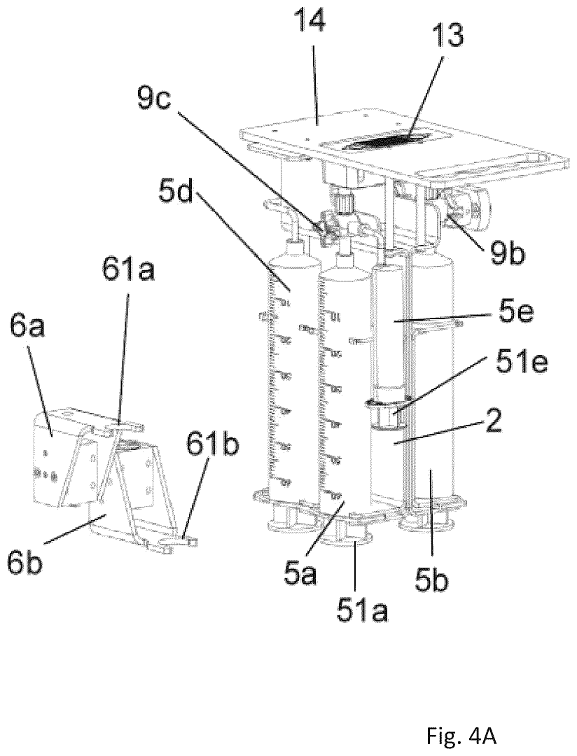

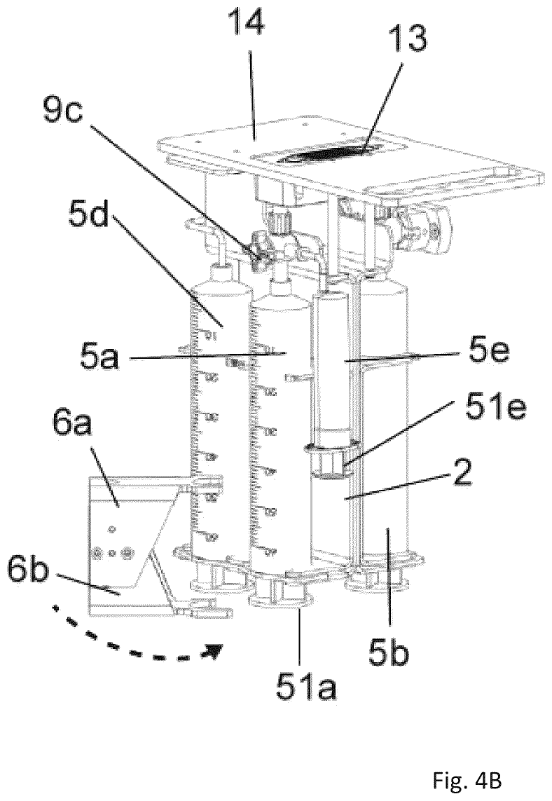

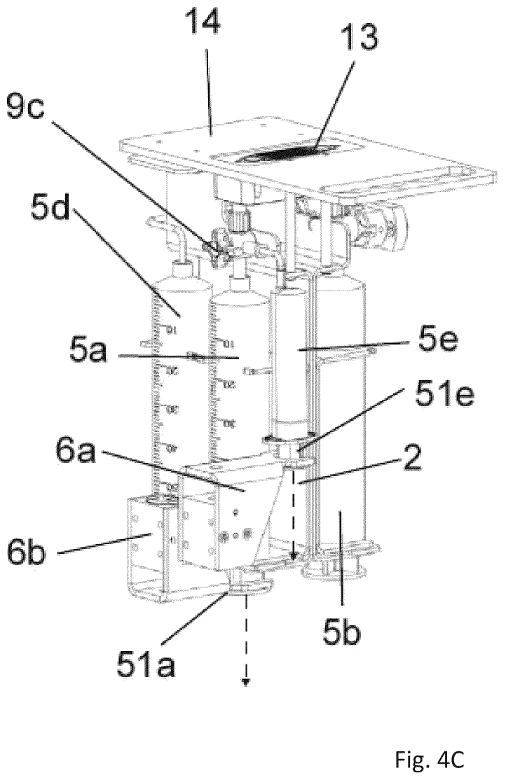

FIGS. 4A, B, C show a carrier cassette including elements of the main module;

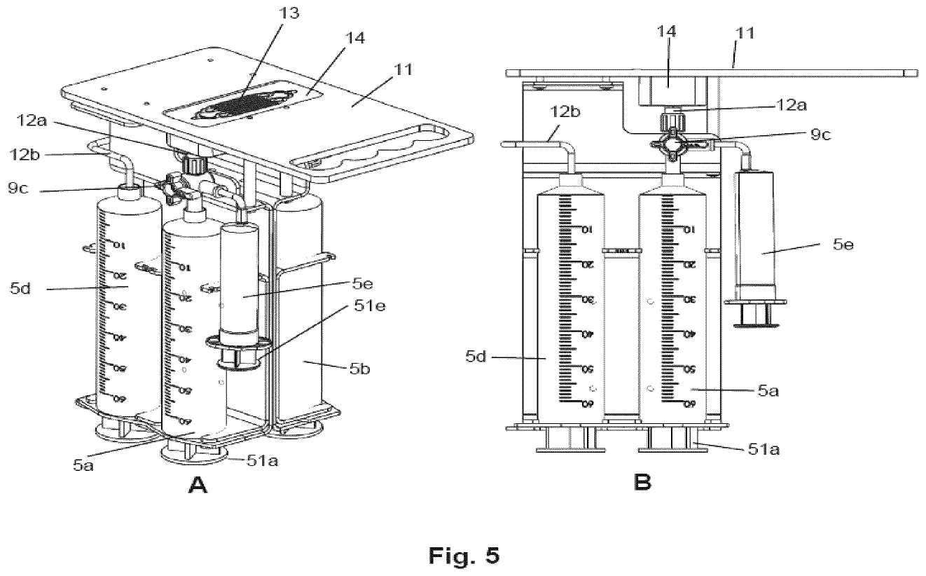

FIGS. 5A, B show a carrier cassette;

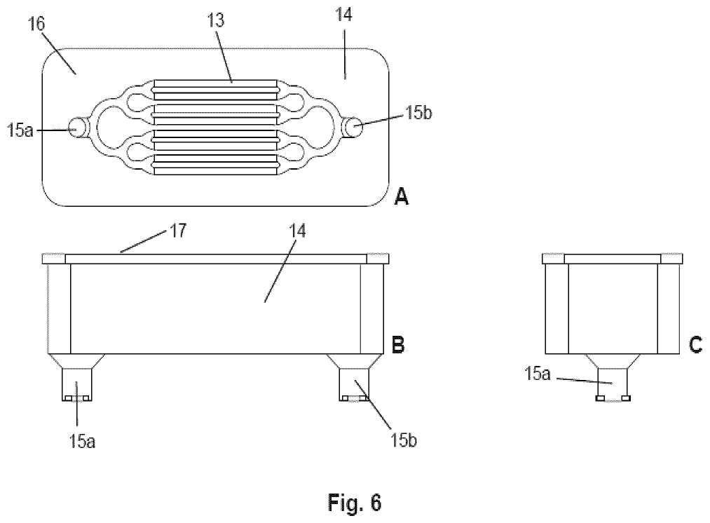

FIGS. 6A, B show a fluid chip;

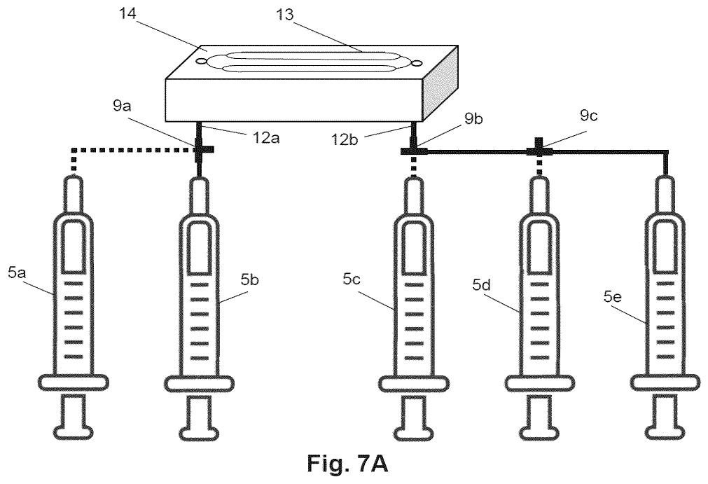

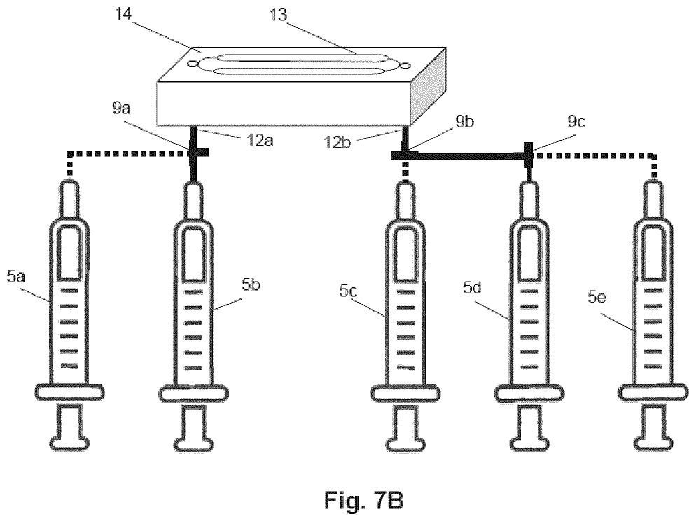

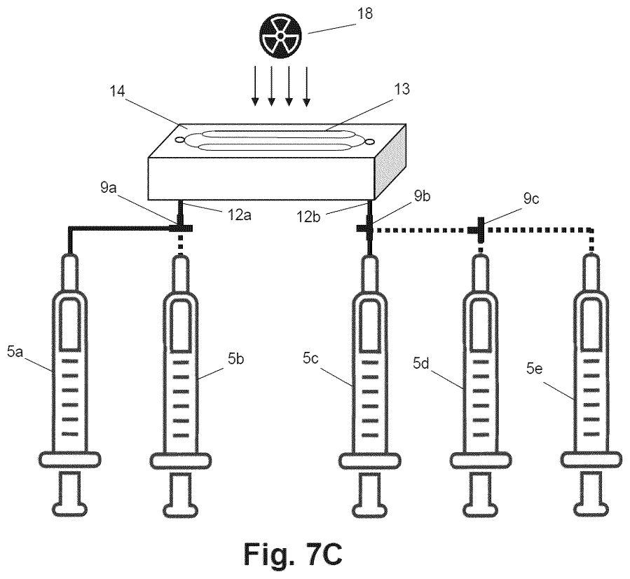

FIGS. 7A, B, C show an exemplary schematic device in which a method for irradiating a fluid can be carried out;

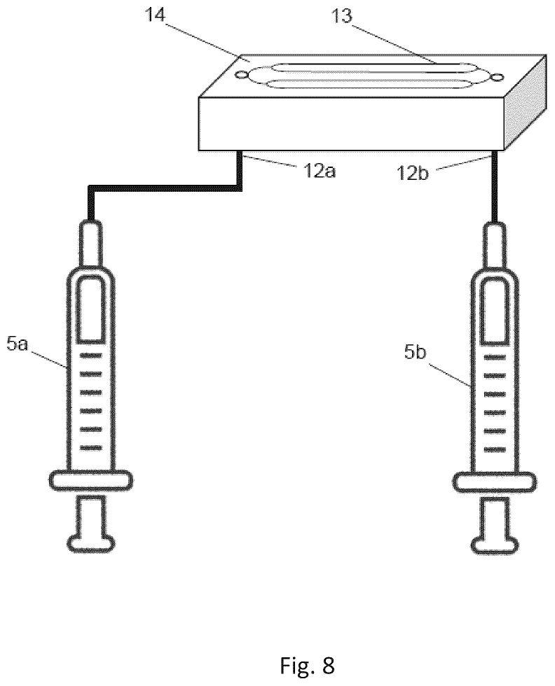

FIG. 8 shows an exemplary schematic device in which a method for irradiating a fluid can be carried out;

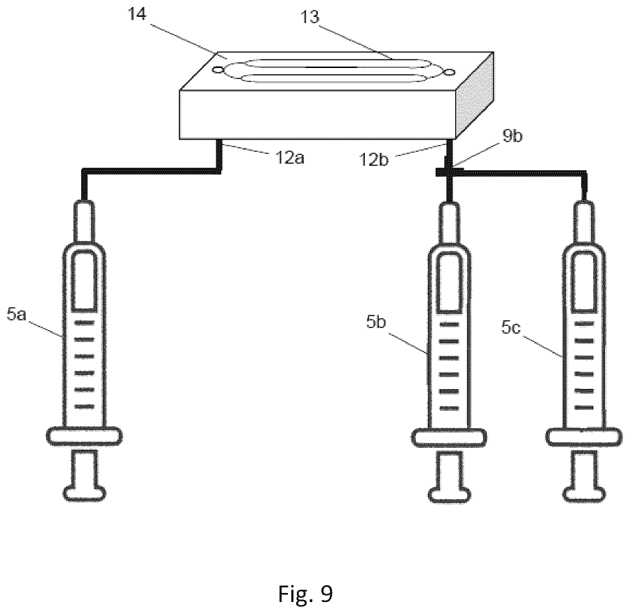

FIG. 9 shows an exemplary schematic device in which a method for irradiating a fluid can be carried out; and

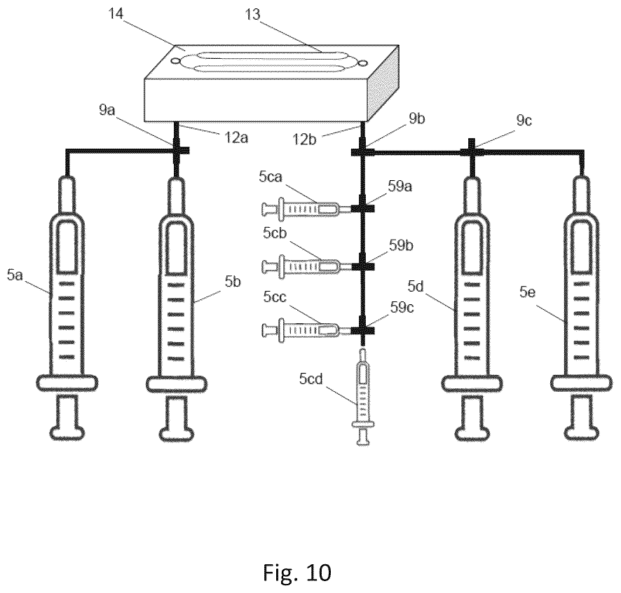

FIG. 10 shows an exemplary schematic device in which a method for irradiating a fluid can be carried out.

FIGS. 1A, B, C show a modular irradiation device according to the invention, comprising a main module 1 and a carrier cassette 2. In FIG. 1A, the carrier cassette 2 is not arranged in the main module and therefore not shown. In FIGS. 1B and C, the carrier cassette 2 is inserted into a receptacle 3 of the main module. FIGS. 2A, B and C each show the irradiation device of FIG. 1 in a side view. The carrier cassette 2 has an exposure surface 4 on which the at least one irradiation line 13 runs, which is apparent in FIGS. 3, 4 and 5. A fluid to be irradiated can be conducted through the irradiation line 13.

The carrier cassette 2 furthermore comprises at least one pump 5a, 5b, which is connected via fluid lines to the irradiation line 13. In addition to the receptacle 3, into which the carrier cassette 2 can be inserted so as to be removable without destruction, the main module 1 comprises at least one pump actuator 6a, 6b for each of the pumps 5a, 5b, by way of which the corresponding of the pumps 5a, 5b can be actuated when the carrier cassette 2 is inserted into the receptacle 3. In the example shown in FIGS. 1 and 2, two pumps 5a, 5b are provided, which are configured as syringes here. Accordingly, the main module 2 comprises the actuators 6a, 6b, which are linear actuators here.

FIGS. 1A and 2A show the main module in each case without the inserted carrier cassette 2. FIGS. 1B and 2B show the main module 1 in each case with the inserted carrier cassette 2. In these examples, the main module 1 comprises two foldable side parts 7a, 7b, which comprise valve actuators 8a, 8b and the linear actuators 6a and 6b. The carrier cassette 2 moreover comprises valves including valve stopcocks 9a, 9b, by which a fluid flow between the pumps 5a, 5b and the irradiation line 13 can be controlled. The linear actuators 6a, 6b and the valve actuators 8a and 8b are arranged at the main module 1 so as to engage on the pumps 5a, 5b and the valve stopcocks 9a, 9b, so that these can be actuated, when the side parts 7a, 7b are being closed after the carrier cassette 2 has been inserted. In the example shown in FIGS. 1C and 2C, the pump 5a is operated by the actuator 6a, while the pump 5b is not actuated, but serves as a reservoir for fluid. The actuator 6b actuates a pump (not shown in the figures) that is arranged behind the pump 5a.

The pumps 5a, 5b are configured as syringes here, each including a plunger running in a cylindrical cylinder. In the example shown, the syringes are arranged having a vertical plunger movement direction and open with the outlet openings thereof into the valves 9a, 9b at the upper end.

The main module 1 is configured as a linkage including four parallel rods, which are vertically positioned and carry a main module surface 10, which closes the main module toward the top. The carrier cassette 2 has an exposure surface 11, which is coplanar with respect to the main module surface 10 when the carrier cassette 2 is inserted into the receptacle 3. The foldable side parts 7a, 7b are arranged at the parallel rods of the main module 1 and rotatable thereabout so as to be folded into the closed state shown in FIGS. 1C and 2C.

FIGS. 3A, 3B and 3C, by way of example, show a carrier cassette 2 as shown in FIGS. 1 and 2 in detail. The carrier cassette comprises four pumps 5a, 5b, 5c, 5d here, of which three are visible. The pumps are again configured as syringes. The pumps 5b and 5c are connected via valves 9a and 9b and a fluid line 12 to an irradiation line 13, which is configured as a channel structure in a fluid chip 14 here. The carrier cassette comprises a support structure 15 here, in which the syringes 5a, 5b, 5c, 5d are removably inserted. The cylinder axes of the syringes 5a, 5b, 5c, 5d are situated parallel to one another.

The valves 9a, 9b are three-way valves here, which can be adjusted by valve stopcocks. FIGS. 3A, 3B, 3C show valve actuators 8a, 8b, which via coupling elements can engage on stopcocks of the valves 9a, 9b and can rotate these. The actuators 8a, 8b are rotating actuators. The actuators 8a, 8b are not part of the carrier cassette 2, but are part of the main module 1, the other components of which are not shown in FIG. 3 for the sake of clarity. FIGS. 3A, 3B and 3C show the valve actuators 8a, 8b in different positions relative to the valves 9a, 9b. These positions form the closing of that side part of the foldable side parts 7b at which the valve actuators 8a, 8b are arranged. FIG. 3C shows the closed state as well as, indicated by arrows, the rotating actuation of the valves 9a, 9b by the valve actuators 8a, 8b. The valve actuators 8a, 8b transmit the torque onto the valves 9a, 9b in a form-locked manner. In the example shown, the coupling elements of the valve actuators 8a, 8b can have a negative shape of the stopcocks of the valves 9a, 9b. However, it shall be noted that the valves can also be adjusted pneumatically, hydraulically, electrically, magnetically or in another manner. The use of rotatable valves is also only an exemplary option.

FIGS. 4A, 4B, 4C show the carrier cassette shown in FIG. 3 rotated 90° about a vertical axis. As a result, the pump 5d, which is not apparent in FIG. 3 and which is arranged next to the other pumps 5a, 5b, 5c parallel thereto, becomes visible. Additionally, a valve 9c is apparent here, via which the pump 5a is connected to the irradiation line 13. Unless stated otherwise here, the description for FIG. 3 also applies to FIG. 4.

In addition to the carrier cassette 2, FIG. 4 shows elements 6a, 6b that are part of pump actuators and therefore part of the main module 1. Of the main module 1, only the components of the valve actuators 6a, 6b that engage on the pumps are shown here, while all other components of the main module 1 are hidden for the sake of clarity. FIGS. 4A, 4B and 4C show the positions of the pump actuators 6a, 6b relative to the carrier cassette 2 as the foldable side part 7a of the main module is being closed.

The pump actuators 6a and 6b each have a recess 61a and 61b. The syringe 5a has an end face 51a that protrudes over a plunger rod of the plunger of the syringe 5a. The smaller syringe 5e accordingly has an end face 51e that projects beyond the plunger of this syringe. Over the course of FIGS. 5A to 5C, the side part 7a is being closed, and the actuators 6a, 6b move toward the syringes 5e and 5a. In the closed state shown in FIG. 4C, the opening 61a of the actuator 6a engages behind the end face 51e of the syringe 5e so that the syringe can be filled by the actuator 6a, as is indicated by the arrow in FIG. 4C. At the same time, the recess 61b of the actuator 6b engages behind the end face 51a of the syringe 5a so that the syringe 5a can be filled by this actuator, as is identified in FIG. 4C by the corresponding arrow.

FIGS. 5A and 5B show the carrier cassette shown in FIGS. 3 and 4 again in an enlarged form, in a perspective view and in a side view. The syringe 5a is connected by way of the three-way valve 9c to the fluid line 12a, which, in turn, is connected to a fluid connection of the fluid chip 12 into which the irradiation line 13 is introduced. The syringe 5d is connected via a further fluid line 12b. With respect to the further design, reference shall be made to the description of FIGS. 3 and 4.

FIG. 6, by way of example, shows a fluid chip 14, as it may be used in FIGS. 1 to 5. The fluid chip 14 can be produced as a monolithic block, for example from polyethylene, into which the irradiation line 13 is embossed or cut. The fluid chip 14 has a base surface exposure surface 16 into which the irradiation line 13 is embossed in the form of a channel structure. The channel structure 13 ends at the fluid connections 15a and 15b. Proceeding from the fluid connection 15a, the channel structure initially splits into two channels, which each in turn split into two channels. Each of these then splits yet again into two channels and they open in this way into a total of eight parallel, straight channel sections. At the opposite end, the straight channel sections combine again in pairs until they open into the shared fluid connection 15b. In the example shown, the fluid connections 15a and 15b are guided out of the fluid chip downwardly in a direction that is perpendicular to the base body exposure surface 16, that is, in the direction away from the base body exposure surface 16, and can be connected there to the fluid lines 12a, 12b. In the example shown, the base body exposure surface 16 is covered with a film 17, which seals the channel structure 13 to prevent the egress of fluid onto the base body exposure surface 16.

FIGS. 7A, 7B, 7C and 7D, by way of example, show how a method according to the invention can be carried out in the modular irradiation device of the invention. For the sake of clarity, FIGS. 7A, B, C, D only show the syringes 5a, 5b, 5c, 5d, 5e, the valves 9a, 9b, 9c and the fluid chip 14 including the irradiation line 13. These elements can be configured as shown in FIGS. 1 to 6. The arrangement of the elements in FIG. 7 here shall only be understood to be schematically functional.

The valves 9a, 9b, 9c are three-way valves here, by way of which it is possible to switch which of the syringes 5a to 5e is connected to the fluid chip 14 in a fluid-conducting manner. The syringes 5a and 5b are connected by way of the three-way valve 9a via a first fluid line 12a to a first fluid connection of the fluid chip 14, and the syringes 5c, 5d, 5e are connected by way of the three-way valves 9b and 9c by means of a second fluid line 12b to a second fluid connection of the fluid chip 14.

Hereafter, it shall be assumed that the syringe 5a is used to receive the irradiated fluid, the syringe 5b is used to receive waste fluid, the syringe 5c contains a cell suspension, the syringe 5d contains a cell medium, and the syringe 5e contains a disinfecting agent, such as ethanol, for example.

After the microfluid chip 14 has been produced and sealed, microbes and the like may be present in the irradiation lines 13. The fluid chip 14 should therefore advantageously be disinfected. For this purpose, as shown in FIG. 7A, the three-way valve 9a is switched so as to establish a connection between the waste syringe 5b and the fluid chip 14. Moreover, the three-way valves 9b and 9c are configured so as to close the syringes 5c and 5d and establish a fluid-conducting connection between the syringe 5e and the fluid chip 14. The syringe 5b is now being filled, whereby fluid, this being the disinfecting agent, is suctioned out of the syringe 5e and conducted through the fluid chip 14.

In the next step shown in FIG. 7B, the position of the three-way valve 9a remains unchanged, so that the waste syringe 5b continues to be connected to the fluid chip 14. The three-way valve 9c, by way of which the syringe 5d is connected to the fluid chip, is positioned in such a way that the syringe 5d is connected to the fluid chip 14 in a fluid-conducting manner. The position of the three-way valve 9b at the syringe 5c remains unchanged. The waste syringe 5b now continues to be filled further, as a result of which fluid, this being cell medium here, is suctioned out of the syringe 5d into the fluid chip 14 and through the same.

In the next step shown in FIG. 7C, the valve stopcock 9a at the first fluid line 12a is now positioned in such a way that the syringe 5a is connected to the first fluid line 12a, and thus to the irradiation line 13 in the fluid chip 14, in a fluid-conducting manner. Moreover, the three-way valve 9b is positioned in such a way that the syringes 5d and 5e are cut off the second fluid line 12b, and the syringe 5c including the cell suspension is connected to the irradiation line 13 in a fluid-conducting manner. Moreover, an irradiation source 18 is activated, which emits radiation onto the exposure surface and the irradiation line 13. Meanwhile, the syringe 5a is being filled, whereby cell suspension from the syringe 5c is suctioned through the fluid chip 14 and received in the syringe 5a. After the irradiation has been completed, the three-way valve 9b can optionally be configured in such a way that the syringe 5d is again connected to the irradiation line 13. In this way, by further filling the syringe 5a, the channel structure 13 can be rinsed with cell medium so as to rinse as many of the irradiated cells as possible from the fluid chip 14 into the syringe 5a.

If needed, the fluid chip 14 can subsequently be rinsed with ethanol from the syringe 5e again. The configuration, in turn, corresponds to that shown in FIG. 7A. The syringe 5b is again filled, whereby disinfecting agent is suctioned from the syringe 5e through the fluid chip 14.

FIG. 8, by way of example, shows a very simple embodiment of the invention in which only one syringe 5a for the irradiated sample and one syringe 5b for cell suspension are provided. The syringe 5a is connected via a first fluid line 12a to a fluid connection of the fluid chip 14, and the syringe 5b is connected via a fluid line 12b to a second fluid connection of the fluid chip 14. In this embodiment, it is not necessary for the irradiation device to comprise valves. All that is needed for irradiation is to activate the irradiation source 18 (not shown here) and to then fill the syringe 5a, whereby cell suspension from the syringe 5b is transported through the channel structure 13 into the syringe 5a.

FIG. 9 shows another simple embodiment of the invention, wherein again only one syringe 5a for the irradiated sample is arranged at the first fluid connection of the fluid chip 14 via the fluid line 12a. A syringe 5b including cell suspension on the one hand and a syringe 5c including cell medium on the other hand are connected via the second fluid line 12b and a three-way valve 9b to the second fluid connection. Similarly to what is shown in FIG. 7, cell suspension can initially be suctioned from the syringe 5b through the fluid chip 14 by filling the syringe 5a. For this purpose, the three-way valve is switched so as to establish a connection between the syringe 5b and the fluid connection of the fluid chip 14. Subsequent to the irradiation, the three-way valve 9b can then be switched so as to establish a connection between the syringe 5c including cell medium and the fluid chip 14, while closing the syringe 5b. If the syringe 5a is then filled further, the channel structure 13 of the fluid chip 14 is rinsed with cell medium, and in this way as many cells as possible are removed from the channel structure 13.

FIG. 10 shows a variant of the situation shown in FIG. 7. FIG. 10 differs from FIG. 7 in that, instead of the syringe 5d in FIG. 7, an arrangement of four syringes 5ca, 5cb, 5cc and 5cd is arranged at the corresponding valve 9b, which is connected by way of three-way valves 59a, 59b, 59c to the valve 9b. Different suspensions can be provided in the syringes 5ca, 5cb, 5cc, which can be conducted through the channel structure 13. Using a syringe 5cd, moreover cell suspension can additionally be provided, which can be used to rinse the system connected to the valve stopcock 9b. By positioning the valves 59a, 59b, 59c, the syringes 5ca, 5cb, 5cd and 5cd can be selectively connected to the valve 9b. The method can then be carried out analogously to that shown in FIG. 7.

The invention allows safe and sterile irradiation of fluids. For example, the microfluid chip 14 can be produced from polyethylene as an injection-molded part and subsequently be sealed with a PET/PE film. The sealing with a thin film (for example <60 μm) helps to ensure that only a small portion of the radiation is absorbed by the film. All components that come in contact with the cell suspension can advantageously be designed as disposable parts, in particular the fluid chip 14 and the syringes 5. As a result of the modular concept including a main module and a carrier cassette, it is possible to produce multiple carrier cassettes and to load them in parallel. In this way, the process preparation becomes parallelizable. It is possible to automatically vent the system so as to avoid possible elasticities and changes in flow associated therewith. The paths between the pumps and the irradiation line are preferably kept short, so that no additional elasticities, for example due to silicone hoses, arise, which could result in changes in the flow rate. Moreover, the dead volume can be kept small, which is a major advantage when producing personalized medicine. The layer thickness and the flow rate of the fluid in the irradiation line can be precisely set and controlled.

1-25. (canceled)

26. A modular irradiation device, comprising:
a main module and
at least one carrier cassette,
wherein the at least one carrier cassette comprises:
an exposure surface, on which the at least one irradiation line runs, wherein a fluid to be irradiated is conductible in the at least one irradiation line, and
at least one pump, which is connected via a first fluid line to the at least one irradiation line; and
the main module comprises:
at least one receptacle in which the at least one carrier cassette can be inserted so as to be removable without destruction, and
at least one pump actuator, which is arranged so as to be able to actuate the at least one pump when the at least one carrier cassette is inserted into the corresponding receptacle.

27. The modular irradiation device according to claim 26, wherein the at least one carrier cassette comprises at least one further pump which is connected via a second fluid line to the irradiation line.

28. The modular irradiation device according to claim 26, wherein the pump actuator comprises a pump coupling element, which is arranged so as to become engaged with a movable element of the corresponding at least one pump in a form-locked manner when the cassette is inserted into the main module.

29. The modular irradiation device according to claim 26, wherein
the at least one carrier cassette comprises at least one valve, which is arranged in at least one of the fluid lines and by way of which a fluid flow between the corresponding pump, which is connected via this fluid line to the irradiation line, and the irradiation line can be controlled, and
the main module comprises a respective valve actuator for one, several or all of the at least one valves, the valve actuators are in each case arranged so as to be able to adjust the corresponding valve when the at least one carrier cassette is arranged in the corresponding receptacle.

30. The modular irradiation device according to claim 29, wherein the at least one valve comprises a stopcock by way of which the valve can be adjusted for controlling the flow of fluid,
the corresponding valve actuator comprising a coupling element, which can be coupled to the stopcock in such a way that a force adjusting the valve or a torque adjusting the valve can be exerted on the stopcock by way of the valve actuator, wherein the coupling element is arranged so as to become engaged with the stopcock when the carrier cassette is inserted into the main module.

31. The modular irradiation device according to claim 29, wherein the at least one valve comprises or is a three-way valve having three ports, one of the fluid lines is connected to a first of the ports, the at least one pump is connected to a second of the ports, and the further pump is connected to the third of the ports.

32. The modular irradiation device according to claim 26, wherein the at least one pump comprises a fluid chamber and a plunger, the plunger sealing the fluid chamber in a fluid-tight manner and being displaceable in the fluid chamber,
the at least one pump actuator engaging on the plunger of the pump when the at least one carrier cassette is inserted into the corresponding receptacle, and a force being applicable in a displacement direction of the plunger by way of the pump actuator.

33. The modular irradiation device according to claim 26, wherein the at least one pump is a syringe.

34. The modular irradiation device according to claim 27, wherein the carrier cassette comprises three pumps that are connected to the second fluid line, and two pumps that are connected to the first fluid line.

35. The modular irradiation device according to claim 26, wherein the main module includes a main module surface, and the exposure surface of the carrier cassette and the main module surface are coplanar when the carrier cassette is inserted into the corresponding receptacle of the main module.

36. The modular irradiation device according to claim 26, wherein the main module comprises exactly one actuator for each pump that is connected to one of the fluid lines, the corresponding pump being actuatable by way of the actuator.

37. The modular irradiation device according to claim 27, wherein the main module comprises two movable and/or foldable side parts.

38. The modular irradiation device according to claim 26, wherein
the carrier cassette comprises a fluid chip,
the fluid chip comprising a base body,
the base body including a base body exposure surface in which the at least one irradiation line is formed, the at least one irradiation line being a channel structure including at least one channel,
the base body furthermore comprising a first fluid connection to which the first fluid line is connected, and a second fluid connection to which the second fluid line is connected,
the base body additionally comprising a film, which is arranged on the base body exposure surface and covers the channel structure,
the film sealing the channel structure against egress of fluid onto the base body exposure surface.

39. The modular irradiation device according to claim 38, wherein the channel structure includes a multitude of the channels, which converge at the respective ends thereof in the respective fluid connection.

40. The modular irradiation device according to claim 39, wherein the channels at their ends respectively converge in pairs into combined channels, and the combined channels, in turn, converge in each case in pairs into combined channels until exactly two combined channels converge into one of the fluid connections.

41. The modular irradiation device according to claim 38, wherein the base body is a monolithic block into which the channel structure is embossed and/or cut.

42. The modular irradiation device according to claim 38, wherein the film has a thickness of smaller than or equal to 80 μm.

43. The modular irradiation device according to claim 38, wherein a depth of the at least one channel is smaller than or equal to 300 μm.

44. An irradiation method for irradiating a fluid in a modular irradiation device according to claim 26,
inserting into the main module at least one of the carrier cassettes, and
irradiating the exposure surface of the at least one carrier cassette with an ionizing radiation, while moving the fluid to be irradiated through the at least one irradiation line, and removing the carrier cassette from the main module.

45. The irradiation method according to claim 44, wherein at least one further carrier cassette is inserted into the main module once or several times after the respective preceding carrier cassette has been removed from the main module, and the exposure surface of the at least one further carrier cassette is irradiated with ionizing radiation, while a fluid to be irradiated is moved through the at least one irradiation line of this further carrier cassette.

46. The irradiation method according to claim 44, wherein a disinfecting agent is moved through the channel structure of the fluid chip after the at least one carrier cassette has been inserted into the main module, and before the fluid to be irradiated is moved through the irradiation line.

47. The irradiation method according to claim 44, wherein a cell medium is moved into the channel structure after the at least one carrier cassette has been inserted into the main module, and before the fluid to be irradiated is moved through the irradiation line, and/or after the fluid to be irradiated was moved through the channel structure.

48. The irradiation method according to claim 44, wherein the fluid transport through the irradiation line is effectuated in that the corresponding of the pumps generates negative pressure.

49. The irradiation method according to claim 44, wherein the fluid to be irradiated comprises or is a cell suspension, virus suspension, medium, serum and/or blood sample.

50. The irradiation method according to claim 44, wherein the ionizing radiation is electrons or UV radiation.
